(19) United States
(12) Patent Application Publication
(10) Pub. No.: US 2013/0315832 A1
(43) Pub. Date: Nov. 28, 2013

(54) DEVICE FOR ANALYZING THE EFFECTS OF AT LEAST ONE PARAMETER RELATING TO A VOLATILE SUBSTANCE

(71) Applicants: Alina Valentina Stoian, Braila (RO); José Gregorio Sanchez, Sussargues (FR); Stéphanie Druon, Jonquieres (FR); Hervé Groux, Le Rouret (FR)

(72) Inventors: Alina Valentina Stoian, Braila (RO); José Gregorio Sanchez, Sussargues (FR); Stéphanie Druon, Jonquieres (FR); Hervé Groux, Le Rouret (FR)

(73) Assignee: IMMUNOSEARCH, Le Plan De Grasse (FR)

(21) Appl. No.: 13/885,803

(22) PCT Filed: Nov. 16, 2011
(86) PCT No.: PCT/EP2011/070287

§ 371 Date: Aug. 6, 2013

(30) Foreign Application Priority Data
Nov. 16, 2010 (FR) 1059417

(51) Int. Cl. G01N 33/50

(52) U.S. Cl. CPC G01N 33/5014
USPC 424/9.2; 435/29; 435/287.1; 422/68.1

(57) ABSTRACT

The invention relates to a device for analysing the effects of at least one parameter related to a volatile substance on a living test system, characterised in that it comprises:
an enclosure (1) composed of a first compartment (20) comprising a gas inlet (21) and a gas outlet (22), and a second compartment (30) in which the test system and the volatile substance are located,
the first (20) and second (30) compartments communicating with each other through an opening (11) fitted with a semi-permeable membrane (40) adapted to limit a flow of volatile substance from the second compartment (30) to the first compartment (20) in a determined manner, while allowing a gas flow from the first compartment (20) to the second compartment, (30), and
the second compartment (30) being hermetically sealed such that the only exchanges with an outside medium take place towards the first compartment (20), through the membrane (40).

The invention relates to the field of analysis of the toxicity of products for living beings (humans or animals living in an aquatic or air environment).

In general, these analyses are made using living test systems that may be air or aquatic animals, plants, algae or even cultures of animal, vegetable or bacterial cells. All of these test systems breathe and live by exchange of oxygen and carbon dioxide in air and therefore cannot survive in hermetically closed confinements for long periods.

Regardless of the procedures envisaged for problems as varied as irritation, sensitisation and ecotoxicity, different in vitro tests are usually carried out bringing these biological test systems into the presence of natural or synthetic chemical substances for which the parameters of interest such as toxicity are to be evaluated. A series of metabolic disturbances is then observed, for example the variation in the expression of biomarkers of the biological activity of test systems, or their mortality and/or metabolic modifications.

Nevertheless, when the substances to be tested are volatile, their concentration varies in time, particularly as a function of their volatility and the location at which the analyses are made. Therefore it is difficult to know the real quantity of substance in contact with the test system at all times. The state of the art usually prefers two solutions to achieve this.

The first consists in carrying out analyses in closed enclosures in order to keep the concentration of the volatile substance constant. Nevertheless, this solution has the disadvantage that it reduces the possible duration of the analysis, at the risk of killing the test systems by depriving them of oxygen.

The second solution consists in carrying out tests on large enclosures, or even open enclosures to guarantee the presence of sufficient air for the test system during the experiment. Tests can then be carried out over longer periods. However, evaporation of the volatile substance causes variations in the concentration that are not controlled and have an influence on the results.

Another possibility is to use chemical libraries listing a number of volatile molecules that are more or less soluble in different solvents. Such chemical libraries that in particular list molecules with a sensitising or irritating potential or that could cause disturbances to fauna and flora in the environment, are used for example in the perfume and cosmetic industries. However, these chemical libraries are not exhaustive and cannot replace analyses on test systems.

Therefore, one objective of the invention is to propose a method and a device for reliably and reproducibly analysing some parameters of chemical substances, particularly volatile substances, on test systems by controlling the concentration of the substance while allowing test systems to breathe throughout the analysis under possibly sterile test conditions, the parameters being particularly the toxicity of volatile substances, their functionality, allergenic potential, mutagenic potential, reprotoxicity potential, toxicity for an aquatic medium, their biodegradation potential, their possibility of transformation into other compounds through a polymerisation, oxidation or reduction reaction, their pharmacological effects (particularly by an analysis of interaction between the volatile compound and the test system usually for a determined time interval with a higher determined concentration than is possible according to the current state of the art), etc.

A secondary objective of the invention is to propose a method of calibrating the analysis device to guarantee that the analysis results are correct.

The invention achieves this by disclosing a device for analysing the effects of at least one parameter related to a volatile substance on a living test system, characterised in that it comprises:
an enclosure composed of a first compartment comprising a gas inlet and outlet, and a second compartment in which the test system and the volatile substance are contained,
the first and second compartments communicate with each other through an opening fitted with a semi-permeable membrane adapted to limit a flow of volatile substance from the second compartment to the first compartment in a determined manner, while allowing a gas flow from the first compartment to the second compartment, and
the second compartment being hermetically sealed such that the only exchanges with an outside medium take place towards the first compartment through the membrane.

Some preferred but non-limitative aspects of the analysis device according to the invention are:

the parameter is the toxicity of the volatile substance;

the gas comprises 5% carbon dioxide and 95% relative humidity;

the volatile substance is mixed so as to obtain a mother solution and the device also comprises a zone adapted to contain said mother solution;

the fluid used for obtaining the mother solution is distilled water or extra pure water;

the mother solution is introduced through a valve; and

the membrane is fixed to the opening through a gasket.

According to a second aspect, the invention discloses a method for analysing the effects of at least one parameter related to a volatile substance on a living test system using a device according to the invention, characterised in that it comprises the following steps:
mix the volatile substance with a fluid so as to obtain an homogeneous mother solution,
place the membrane on the opening, in a sealed manner,
add the mother solution into the second compartment and close the second compartment so that the only exchanges with an outside medium take place towards the first compartment through the membrane,
transfer a gas flow into the first compartment for a determined period,
determine the variation in the concentration of the volatile substance in the second compartment with time, and
starting from the variation of the concentration of the volatile substance with time, analyse the test system and use the analysis to determine the parameter for the volatile substance.

Preferred but non-limitative aspects of the analysis method include the following:

it also comprises a step during which the enclosure is brought to a determined temperature before the mother solution is added;

the mother solution and the gas flow scavenging the first compartment are added at the determined temperature of the enclosure;

the determined temperature is about 37° C.+/−1° C.;

the variation in the concentration of the volatile substance in time is determined as a function of at least one of the following parameters:
the transfer area of the mother solution into the second compartment,
the volume of the second compartment,
the depth of the mother solution in the second compartment,
the volume of the mother solution and/or the gas volume present in the second compartment,
the temperature of the mother solution or the gas present in the second compartment,
the pressure in the first compartment and/or the second compartment,
the composition of the gas scavenging the first compartment,
the gas flow in the first compartment,
properties of the volatile substance, and,
properties of the membrane;

properties of the volatile substance comprise at least one of the elements in the following group: its sharing coefficient, its coefficient of diffusion in water, its coefficient of diffusion in air, its viscosity in the liquid phase, its viscosity in the vapour phase, its density in the liquid phase and its density in the vapour phase;

properties of the membrane comprise at least one of the elements in the following group: its porosity, the pore size and its tortuosity, and

it also comprises a preliminary step to homogenise the mother solution.

Other characteristics, purposes and advantages of this invention will become clear after reading the following detailed description with reference to the appended drawings given as illustrative examples and in which:

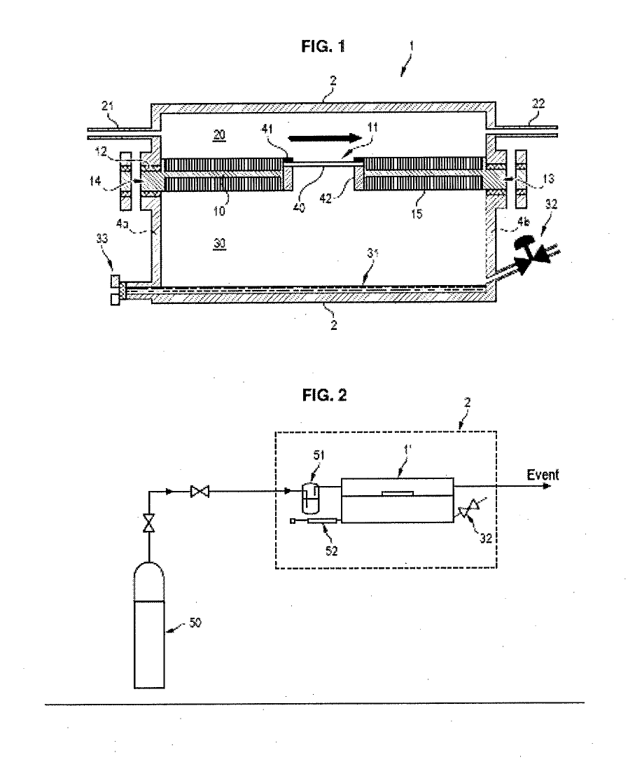

FIG. 1 shows a sectional view of one embodiment of the device according to the invention,

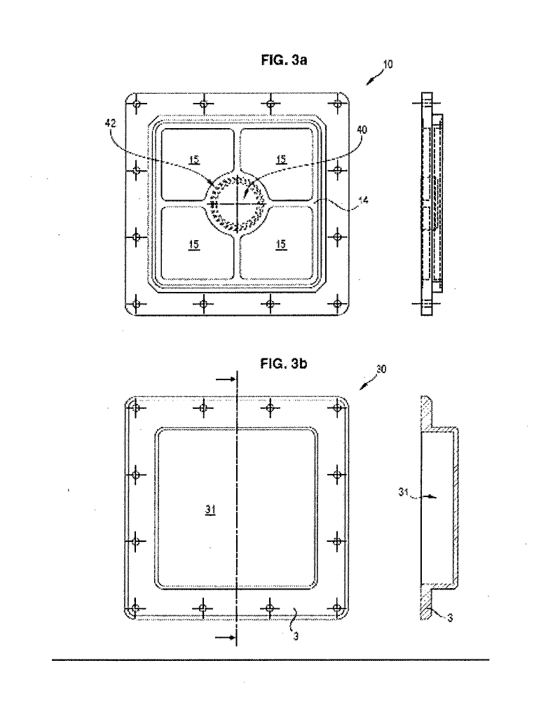

FIG. 2 shows an example installation of the device during the analysis, and

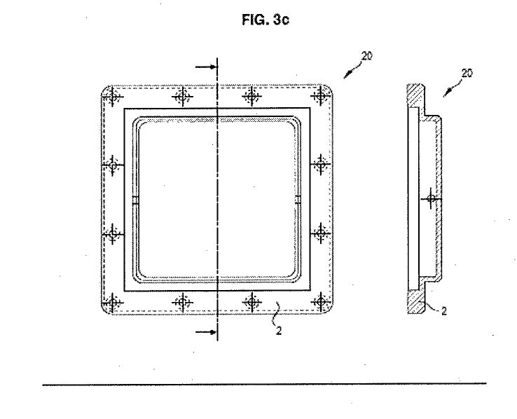

FIGS. 3a, 3b and 3c show front and sectional views of the separation element between the two compartments, the first compartment and the second compartment respectively.

We will now describe a device for analysing the effects of at least one parameter related to a substance on a living test system according to the invention.

FIG. 1 shows one embodiment of a device. The device comprises an enclosure 1, preferably comprising a top wall 2 and a bottom wall 3 connected to each other in a sealed manner by four sidewalls 4a, 4b, 4c, 4d. The walls 2-4d may be made particularly from stainless steel.

The enclosure 1 also comprises an internal separation wall 10 that extends between two opposite sidewalls of the enclosure so as to define two compartments 20, 30.

In this case, a first compartment 20 extends in the upper part of the enclosure 1 between the top wall 2 and the separation wall, while the second compartment 30 extends between the separation wall 10 and the bottom wall 3. However, this is not limitative; the separation wall 10 may extend between the top wall and the bottom wall so as to vertically separate the two compartments 20 and 30.

The separation wall 10 is preferably solid, except for an opening 11 with determined dimensions through which compartments 10 and 20 communicate with each other.

The dimensions of the opening 11 depend on the analysis conditions and can vary. Consequently, the dimensions of the opening 11 may be variable (such as a diaphragm).

As a variant, the separation wall 10 is removable so that it can be replaced by a new wall 10 with an opening 11 with different dimensions. For example, a first separation wall may comprise an opening 11 with a 39 mm diameter while a second wall may have an opening 11 with a 20 mm diameter.

The separation wall 10 can then be fixed in the enclosure removably and in a sealed manner, for example by means of a gasket 12 and possibly by tightening of screws and nuts 13.

The separation wall 10 may be made of stainless steel. As a variant, the separation wall 10 may be made lighter by using a stainless steel plate 14 enclosed between two Teflon plates 15.

A membrane 40 covers the opening 11.

Advantageously, the membrane 40 is fixed in a sealed manner onto the opening 11, for example through a stainless steel support 42 and a gasket 41. In this case, the gasket 41 is a flat gasket such as a flat rubber gasket, a flat VITON® gasket or a flat TEFLON® gasket.

According to one preferred embodiment, the membrane 40 is semi-permeable, in other words it allows the passage of fluids (in this case air) from one of the compartments (in this case the upper compartment 20), and limits or prevents the passage of fluids from the other compartment (in this case vapours of the volatile substance from the lower compartment 30). More precisely, and as will be seen in the remainder of the description, the function of the membrane 40 is to limit or prevent the passage of volatile substances from the lower compartment 30, while allowing exchanges of oxygen and carbon dioxide from the upper compartment 20 or the lower compartment 30.

For example, the membrane 40 can be a commercial membrane like those usually used in the field of ventilation for gas filtration and sterilisation.

The membrane 40 is preferably chosen so as to have pore diameters smaller than or equal to 0.22 μm, in order to sterilise air passing through it from the upper compartment 20 to the lower compartment 30.

Typically, it may be a Durapore® membrane marketed by the Millipore Company. This membrane is made from polyvinylidene fluoride and its characteristics defined by the manufacturer are pore size=0.1 μm, membrane diameter between 5 and 47 mm, membrane thickness=125 μm, porosity (corresponding volume of pores in the membrane)=70%, air flow=3 L/min/cm²), and tortuosity between 2 and 4.

It may also be an Emflon® membrane marketed by the Pall Corporation Company. This membrane is made from polytetrafluoroethylene (PFTE) and its characteristics defined by the manufacturer are pore size=0.02 μm, membrane thickness=0.22 mm+/−0.1 mm, porosity=70%, air flow=62.30 cm3/min/cm²/psi, membrane diameter between 5 and 100 mm and tortuosity between 2 and 4.

Other membranes made of porous polymer, porous metal or porous ceramic could also be used provided that they are semi-permeable.

Note that the use of these membranes creates a critical zone at the opening 11 in which particles of the volatile substance can accumulate without being able to pass through to the upper compartment 20. It can then be said that this accumulation forms a sort of “barrier” reinforcing the resistance formed by the membrane 40, without being able to prevent air passage from the upper compartment 20.

If required, the size of the membrane 40 can be adjusted as a function of the diameter of the opening 11 and the support provided on the separation wall 10.

The first compartment 20, referred herein as the upper compartment, comprises an inlet and an outlet preferably extending between two opposite walls 4a, 4b of the enclosure 1, and adapted to enable a gas fluid to pass through the compartment 20 from one wall 4a to the other 4b. The flow of the gas fluid can optionally be controlled by valves placed at the inlet 21 and the outlet 22 respectively of the first compartment 20. For example, it may be of the order of 17.64 L·h⁻¹.

The second compartment 30, herein referred to as the lower compartment, comprises a zone 31 adapted to contain a solution. In particular, it can be a receptacle, etc. FIG. 3b shows the zone 31 as a recess formed in the bottom wall 3 of the enclosure 1.

Advantageously, the zone 31 is in fluid communication with a valve 32 through which fluid can be added into the lower compartment 30, particularly gas. The lower compartment may also comprise a septum 33, typically made of PFTE or rubber, in fluid communication with the zone 31 and adapted so that the fluid can be sampled if required (particularly for a concentration measurement, etc.). In this way, when the valve 32 and the septum 33 are closed, the lower compartment 30 is sealed so that the only exchanges with the medium outside the compartment 30 take place through the membrane 40 to the upper compartment 20.

We will now describe a method for analysing the effects of at least one parameter related to a volatile substance on a test system.

In the following, we will describe the method especially in its application to the analysis of the toxicity of volatile substances on animal, vegetable or bacterial cell cultures. Furthermore, the enclosure 1 chosen in our example is about 5 cm high (between the top face 2 and bottom face 3) and its width and depth are about fifteen centimetres. The ratio between the volume of the second compartment 30 and the total liquid volume is variable but it may for example be between 9 and 10, and the ratio between the total volume of the compartment 30 and the transfer area of the fluid retained in the zone 31 is preferably of the order of 3. However, this is not limitative.

Before the test, it is preferable to bring the temperature of the enclosure 1 to a fixed determined temperature, for example 37° C.+/−1° C., so as to thermostatically control it. More generally, the temperature of the enclosure depends on the type of test being done, the volatile substance and the test system, but preferably remains constant throughout the test. A type of opening 11 of the corresponding separation wall 10 is then chosen, and the wall is fixed in the enclosure in a sealed manner so as to form the two compartments 20, 30.

A semi-permeable membrane 40 is fixed and sealed on the separation wall 10, either after or before placement of the separation wall 10 in the enclosure 1), using a gasket 41.

Furthermore, the enclosure is preferably kept at a determined temperature throughout the tests, so as to keep the temperature in enclosure 1 and the elements contained in it constant throughout the tests. For example, the enclosure is placed in a warming cabinet maintained at 37° C.

The enclosure 1 is then ready for use.

A mother solution is prepared comprising a fluid with which the volatile substance to be tested is mixed.

The fluid may be water, preferably ultra pure water (for example Millipore Q-Guard® 1 ultra pure water) or distilled water, the purpose being to limit contamination of the fluid used.

The volatile substance is then mixed in water at a dose less than or equal to the solubility of said substance in water. For example, it is mixed at a concentration of 95% of the solubility limit of the substance in water.

According to one preferred embodiment, the solution is also homogenised, for example in an ultrasound bath regulated to the same temperature as the enclosure, in this case 37° C. The mother solution can then be stored until it is used in a sealed receptacle preferably placed in a warming cabinet also thermostat controlled at 37° C.

The gas preferably comprises 5% carbon dioxide (CO₂) at 95% relative humidity, so as to simulate the conditions of an incubator used for the cell culture. For example, a gas cylinder 50 containing a mix of air with 5% CO₂ such as the “Crystal” mix by Air Liquide, is connected to the inlet 21 of the upper compartment 20 through an air humidifier 51 adapted to adjust the relative humidity of air to 95%.

The air thus humidified is then introduced into the upper compartment 20, preferably at a flow such that the flow in the upper compartment 20 is laminar. The air flow may be regulated particularly using valves at the inlet 21 and the outlet 22 of the compartment 20.

Advantageously, air inlet into the upper compartment 20 is at the same temperature as the enclosure 1, in this case 37° C. and at atmospheric pressure.

The mother solution is then added into the zone 31 of the lower compartment 30, preferably at a temperature of 37° C., through the valve 32. This is done particularly using a syringe 52 provided with a luer lock connection and a metal needle at its end.

Furthermore, the gas present in the lower compartment 30 may be ambient air, preferably at the same temperature as the remainder of the device, in this case 37° C.

The test system can then be placed in the lower compartment 30. For example in the case of cellular cultures, cultures are introduced into the zone 31 with the mother solution.

In the case of animals, they are placed in the lower compartment, for example by opening the enclosure 1 to obtain access to it. To achieve this, the enclosure comprises a removable part, for example a window in a wall providing access to the lower compartment 30.

Obviously, the order of the steps mentioned above is not limitative. Typically, the mother solution containing the volatile compound can be added after the test system has been placed in the enclosure 1.

As a variant, the upper compartment 20, the lower compartment 30 and the wall 10 are removable.

The test system is taken out of the lower compartment after a determined time, depending particularly on the type of the volatile substance being tested, and tests are made on it to evaluate at least one parameter of the volatile substance, typically its toxicity.

The duration of the test in the enclosure may be between 10 minutes and 48 hours.

The variation in the concentration of the substance with time is determined in order to better determine the impact of the volatile substance on the test system.

This variation is determined particularly from all or some of the following characteristics: the transfer area of the mother solution into the lower compartment 30, the volume of the lower compartment 30, the depth of the mother solution in the lower compartment 30, the volume of the mother solution and/or the volume of gas present in the lower compartment 30, the temperature of the mother solution or the gas present in the lower compartment 30, the pressure in the upper compartment 20 (in this case the atmospheric pressure), the composition of gas scavenging the upper compartment 20, the gas flow in the upper compartment 20, properties of the volatile substance and properties of the membrane 40.

Properties of the volatile substance that can be considered include particularly its sharing coefficient, its coefficient of diffusion in water, its coefficient of diffusion in air, its viscosity in the liquid phase, its viscosity in the vapour phase, its density in the liquid phase, or its density in the vapour phase.

Nevertheless, the model of the variation in the concentration depends on the membrane 40 used during the test. Therefore, the properties of the membrane 40 also have to be taken into account, including particularly its porosity, pore size and tortuosity.

In particular, these properties may be verified before the analysis method according to the invention is used, so as to take account of precise values in the model of how the concentration of volatile substance changes in the lower compartment.

For example, the porosity of the membrane 40 may be measured by means of a mercury porosity meter, its tortuosity can be measured by means of a gas permeation experiment and its thickness can be measured using a scanning electron microscope.

Measurements of the concentration of the volatile substance in the mother solution can be made at regular intervals in order to validate the model. To do this, a sample (typically 1 μL so as not to modify the thermodynamic equilibrium in the lower compartment 30) of the mother solution may be taken at regular intervals and the concentration may be measured by gas phase chromatography, and the variation in the measured concentration can then be compared with the model results. When the system is in equilibrium in the lower compartment 30, the concentration of the volatile substance in the mother solution is constant. Consequently, a reduction in the concentration in the mother solution implies a transfer of some of the volatile system through the membrane 40.

1. Device for analysing the effects of at least one parameter related to a volatile substance on a living test system, characterised in that it comprises:
an enclosure composed of a first compartment comprising a gas inlet and a gas outlet, and a second compartment in which the test system and the volatile substance are contained,
the first and second compartments communicate with each other through an opening fitted with a semi-permeable membrane adapted to limit a flow of volatile substance from the second compartment to the first compartment in a determined manner, while allowing a gas flow from the first compartment to the second compartment, and
the second compartment being hermetically sealed such that the only exchanges with an outside medium take place towards the first compartment, through the semi-permeable membrane.

2. Analysis device according to claim 1, wherein the parameter is the toxicity of the volatile substance.

3. Analysis device according to claim 1, wherein the gas comprises 5% carbon dioxide and 95% relative humidity.

4. Analysis device according to claim 1, wherein the volatile substance is mixed with a fluid so as to obtain a mother solution, and in that the device also comprises a zone adapted to contain said mother solution.

5. Analysis device according to claim 4, characterised in that the fluid used for obtaining the mother solution is distilled water or extra pure water.

6. Analysis device according to one of claims 4 or 5, wherein the mother solution is introduced through a valve.

7. Analysis device according to claim 1, wherein the semi-permeable membrane is fixed to the opening through a gasket.

8. Method for analysing the effects of at least one parameter related to a volatile substance on a living test system using a device according to claim 1, comprising the following steps:
mix the volatile substance with a fluid so as to obtain an homogeneous mother solution,
place the semi-permeable membrane on the opening in a sealed manner,
add the mother solution into the second compartment and close the second compartment, so that the only exchanges with an outside medium take place towards the first compartment, through the semi-permeable membrane,
transfer a gas flow into the first compartment for a determined period,
determine the variation in the concentration of the volatile substance in the second compartment, and
starting from the variation of the concentration of the volatile substance with time, analyse the test system and use the analysis to determine the parameter for the volatile substance.

9. Method according to claim 8, further comprising a step during which the enclosure is brought to a determined temperature before the mother solution is added.

10. Method according to claim 9, wherein the mother solution and the gas flow scavenging the first compartment are added at the determined temperature of the enclosure.

11. Method according to claim 10, wherein the determined temperature is about 37° C.+/−1° C.

12. Method according to claim 8, wherein the variation in the concentration of the volatile substance in time is determined as a function of at least one of the following parameters:
a transfer area of the mother solution into the second compartment,
a volume of the second compartment,
a depth of the mother solution in the second compartment,
a volume of the mother solution and/or the gas volume present in the second compartment,
a temperature of the mother solution or the gas present in the second compartment,
a pressure in the first compartment and/or the second compartment,
a composition of the gas scavenging the first compartment,
a gas flow in the first compartment,
properties of the volatile substance, and,
properties of the semi-permable membrane.

13. Method according to claim 12, wherein the properties of the volatile substance comprise at least one of the elements in the following group: its sharing coefficient, its coefficient of diffusion in water, its coefficient of diffusion in air, its viscosity in the liquid phase, its viscosity in the vapour phase, its density in the liquid phase and its density in the vapour phase.

14. Method according to one of claims 12 or 13, wherein properties of the semi-permeable membrane comprise at least one of the elements in the following group: its porosity, the pore size and its tortuosity.

15. Method according to claim 8, further including a preliminary step to homogenise the mother solution.
